(19) United States
(12) Patent Application Publication
(10) Pub. No.: US 2006/0229641 A1
(43) Pub. Date: Oct. 12, 2006

(54) GUIDANCE AND INSERTION SYSTEM

(76) Inventors: Rajiv Gupta, Wayland, MA (US); Steven R. H. Barrett, Cambridge (GB); Nevan C. Hanumara, Kingston, RI (US); Jo-Anne O. Shepard, Dover, MA (US); Alexander H. Slocum, Bow, NH (US); Conor James Walsh, Dublin (IE)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/307,231

(22) Filed: Jan. 27, 2006

Related U.S. Application Data
(60) Provisional application No. 60/647,867, filed on Jan. 28, 2005.

(51) Int. Cl. A61B 19/00

(52) U.S. Cl. 606/130

(57) ABSTRACT

Methods and devices are provided for guiding and inserting a tool into an object, such as tissue. In an exemplary embodiment, a guidance and insertion device is provided that can be remotely controlled to adjust an insertion trajectory of a tool, and to advance the tool into tissue to a desired penetration depth. The tool can be, for example, a biopsy device, a brachytherapy device, or a lumpectomy device. The device can be configured for use with an imaging apparatus, such as computed tomography (CT) images, to allow the device and tool to be operated while viewing the device positioned in relation to a target surgical site. The device can also be configured to be positioned directly on a patient, so as to passively compensate for respiratory chest motion, and it can include features to passively compensate for needle oscillation. In other exemplary embodiments, the device can be entirely disposable.

### CROSS REFERENCE TO RELATED APPLICATIONS

The present invention claims priority to U.S. Provisional Application No. 60/647,867, which was filed on Jan. 28, 2005 and entitled “Needle Guidance System for Percutaneous Lung Biopsy,” which is hereby incorporated by reference in its entirety.

### FIELD OF THE INVENTION

The present invention relates to methods and devices for guiding and inserting a tool into a target surgical site.

### BACKGROUND OF THE INVENTION

Needle biopsies are performed to retrieve a sample of human tissue or fluid for histological and chemical analysis. A 14 to 20 gauge needle is inserted through a patient's skin until it reaches the target surgical site from where the sample is extracted. When it is desirable to target a particular region, such as a lesion caused by lung cancer, the procedure is often carried out under the guidance of computed tomography (CT). This results in an iterative procedure whereby, following an initial scan to target the biopsy site and planning the insertion trajectory, in terms of angle and depth, the needle is incrementally inserted and the patient is repeatedly scanned to verify and adjust the needle position. Often the CT gantry is tilted to coincide with the needle's plane of insertion so that the metallic needle is clearly visible in a single CT scan slice. This interactive procedure necessitates that a doctor and support staff repeatedly shuttle between the radiation-shielded control room (during scanning) and the CT room (when manipulating the needle) and that the patient be moved in and out of the CT scanner's ring to allow access to the insertion site.

Accordingly, there remains a need for improved methods and devices for guiding and inserting a needle or other tools into a patient.

### BRIEF SUMMARY OF THE INVENTION

The present invention provides methods and devices for guiding and inserting a tool into an object, such as a body or tissue. In one exemplary embodiment, a guidance and insertion system is provided having a support base that is adapted to be located with respect to a tissue surface, a carriage that is movably mounted on the support base and that defines a passage, such as an aperture or viewing window, therethrough for receiving a tool, and a driver coupled to the carriage and adapted to drive a tool through the passage and into the object positioned beneath the support base.

While the carriage can be movably coupled to the support base using a variety of techniques, in one exemplary embodiment the carriage is movably mounted to the support base by first and second support arms that are movably coupled to the support base. The first and second arms can have a variety of shapes and sizes, but in one embodiment each support arm can have a substantially arcuate shape, and they can extend substantially transverse to one another. Each arm can also include an opening formed therein. The openings can overlap one another such that a portion of the carriage can extend through the openings in the first and second support arms. In use, the arms can be rotatably coupled to the support base and movement of the first and second support arms relative to one another can move the carriage relative to the support base. Each of the first and second arms can also include a drive socket formed thereon and adapted to couple to a motor for individually moving the first and second support arms relative to the support base. The motor can optionally be configured to be remotely actuated to allow the device to be used simultaneously with an imagining apparatus.

The carriage can have a variety of configurations, but in one embodiment it can include an engagement mechanism slidably disposed thereon and adapted to slidably move to engage a tool extending through the passage. The engagement mechanism can include an opening, such as a cut-out, formed therein and adapted to seat a tool extending through the passage. The cut-out is preferably configured to urge the tool into a predetermined position. The carriage can also include a driver rotatably coupled thereto and adapted to rotate to slide the engagement mechanism within a track formed in the carriage. In one embodiment, the driver can include at least one roller that is adapted to rotate to drive a tool through the passage. In certain exemplary embodiments, the driver includes an active roller that is adapted to couple to a motor for rotating the roller, and a passive roller. The active roller can be coupled to the carriage, and the passive roller can be coupled to an engagement mechanism that is slidably disposed on the carriage and that is adapted to slidably move to engage a tool extending through the passage.

A method for guiding and inserting a tool into an object, such as a patient, is also provided, and in one exemplary embodiment includes positioning a guide system on a tissue surface of a patient such that the guide system is positioned over a target surgical site, and positioning a tool through a viewing window of the guide system. In an exemplary embodiment, the tool can be positioned by penetrating a distal portion of the tool into tissue. The method can also include actuating the guide system to engage the tool positioned within the viewing window, actuating the guide system to adjust a trajectory of the tool while viewing an image of the guide system and the target surgical site, and actuating a driver mechanism on the guide system to advance the tool into tissue and toward the target surgical site. In an exemplary embodiment, the guide system can be actuated to adjust a trajectory of the tool by pivoting at least one arm pivotally coupled to a support base of the guide system. The arm(s) can have a carriage mounted thereon and defining the viewing window extending therethrough. In another exemplary embodiment, the tool can be engaged by slidably moving an engagement mechanism disposed on the carriage to a closed position. The tool can be positioned between an active roller and a passive roller in the engaged position, and the active roller can be actuated to advance the tool. In another exemplary embodiment, an image of the guide system and the target surgical site can be viewed using computer tomography, and the guide system and driver mechanism can be simultaneously actuated remotely, i.e., a distance away from the device.

### BRIEF DESCRIPTION OF THE DRAWINGS

The invention will be more fully understood from the following detailed description taken in conjunction with the accompanying drawings, in which:

FIG. 1A is a perspective view of one exemplary embodiment of a guidance and insertion device;

FIG. 1B is a top view of the guidance and insertion device of FIG. 1A, showing an engagement mechanism in an open position;

FIG. 1C is a top view of the guidance and insertion device of FIG. 1A, showing an engagement mechanism in a closed position;

FIG. 2 is a perspective view of a support base of the guidance and insertion device of FIGS. 1A-1C;

FIG. 3 is a perspective view of a support arm of the guidance and insertion device of FIGS. 1A-1C;

FIG. 4A is a perspective view of a carriage assembly of the guidance and insertion device of FIGS. 1A-1C, showing a carriage, an engagement mechanism, and a driver;

FIG. 4B is an exploded view of the carriage assembly shown in FIG. 4A;

FIG. 5A is a perspective view of the carriage shown in FIGS. 4A and 4B;

FIG. 5B is a bottom view of the carriage shown in FIG. 5A;

FIG. 5C is a cross-sectional view of the carriage shown in FIGS. 5A and 5B;

FIG. 6 is a perspective view of the engagement mechanism shown in FIGS. 4A and 4B;

FIG. 7 is a cross-sectional view of the guidance and insertion device of FIGS. 1A-1C;

FIG. 8A is a side view of a guidance and insertion system, showing the guidance and insertion device of FIGS. 1A-1C and a needle positioned through a viewing window formed in the device;

FIG. 8B is a perspective view of the guidance and insertion system shown in FIG. 8A, showing a viewing window for allowing movement of the needle;

FIG. 8C is a perspective view showing the guidance and insertion system of FIG. 8B with a sliding engagement mechanism moved to a closed position to engage the needle;

FIG. 8D is a top view showing the guidance and insertion system of FIG. 8C with first and second arms of the device pivoted to move a carriage of the device, thereby adjusting an insertion trajectory of the needle;

FIG. 8E is a top view showing the guidance and insertion system of FIG. 8D with the needle advanced by a driver mechanism on the guidance and insertion device;

FIG. 8F is a top view showing the guidance and insertion system of FIG. 8E with the engagement mechanism moved to an open position to release the needle; and

FIG. 8G is a perspective viewing of the guidance and insertion system shown in FIG. 8F.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides methods and devices for guiding and inserting a tool, such as a biopsy device, a brachytherapy device, computer chip, wires, or a lumpectomy device, into tissue. In an exemplary embodiment, a guidance and insertion device is provided that can be remotely controlled to adjust an insertion trajectory of a tool, and to advance the tool into tissue to a desired penetration depth. The device can be configured for use with an imaging method, such as computed tomography (CT), magnetic resonance imaging (MRI), X-ray fluoroscopy, or ultrasound, to allow the device and tool to be operated while simultaneously viewing the device positioned in relation to a target surgical site. The device can also be configured to be positioned directly on a patient, so as to passively compensate for respiratory chest motion, and it can include features to passively compensate for oscillation of the needle or any other tool. In other exemplary embodiments, the device can be entirely disposable.

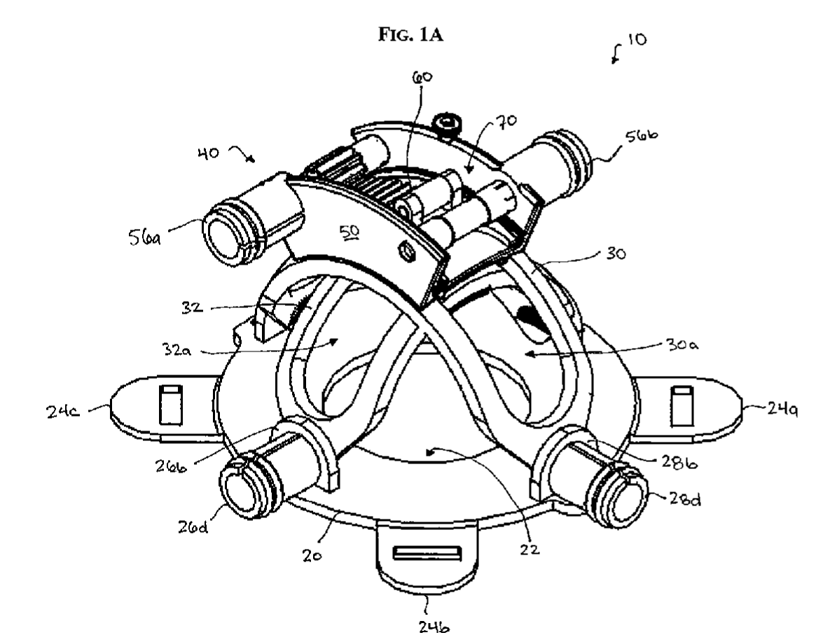

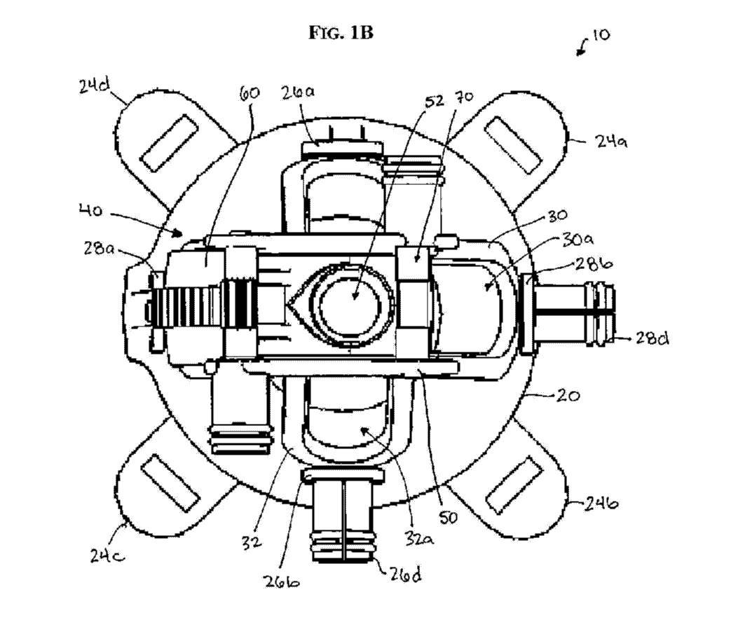

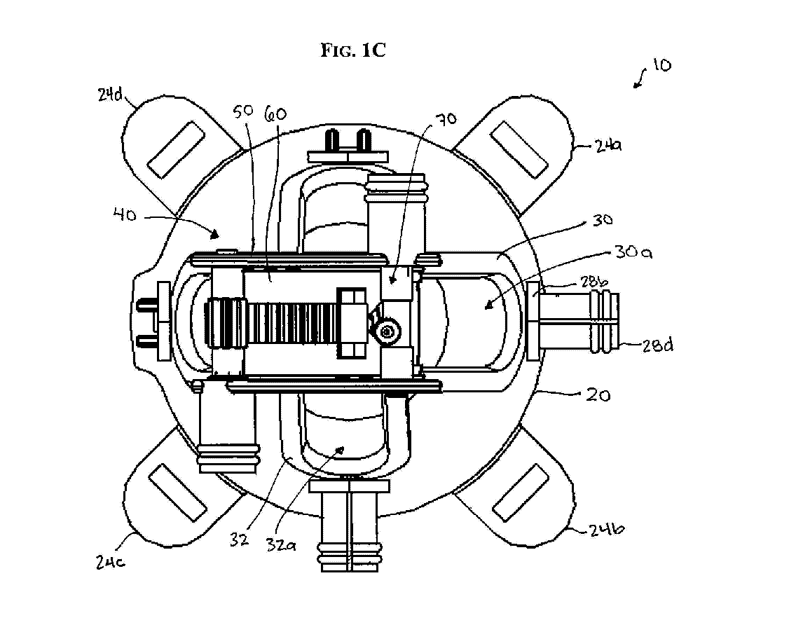

FIGS. 1A-1C illustrate one exemplary embodiment of a guidance and insertion device 10 for guiding and inserting a tool into tissue. In general, the device 10 includes a support base 20 that is adapted to be positioned on a tissue surface. First and second support arms 30, 32 are movably coupled to the support base 20, and each arm 30, 32 includes an opening 32a, 32b formed therethrough. The device 10 can also include a carriage assembly 40 that is movably coupled to the first and second support arms 30, 32. The carriage assembly 40 can include a carriage 50 defining a passage, referred to herein as a viewing window 52 (FIG. 1B), for receiving a tool, an engagement mechanism 60 that is configured to engage a tool extending through the viewing window 52, and a driver mechanism 70 for driving the tool through the viewing window 52. In use, the guidance and insertion device 10 can be positioned on a tissue surface of a patient, and a tool (not shown) can be positioned through the viewing window 52 in the carriage 50. The engagement mechanism 60 can be moved from an open position (FIG. 1B) to a closed position (FIG. 1C) to engage the tool, and the first and second arms 30, 32 can be moved relative to the support base 20, thereby adjusting a position of the carriage 50 and the tool extending therethrough. An image of the device, tool, and target tissue site can be simultaneously used to facilitate positioning of the carriage 50 and thus the tool insertion trajectory. Once the tool is properly positioned, the driver mechanism 70 can be actuated to drive the tool into tissue to a desired penetration depth. Imaging can likewise be used to simultaneously view and facilitate insertion of the tool to a proper depth. In an exemplary embodiment, the device 10 is used to guide and insert a biopsy probe or needle to a target surgical site, such as a tumor. A person skilled in the art will appreciate, however, that the device can be used to guide and insert a variety of tools.

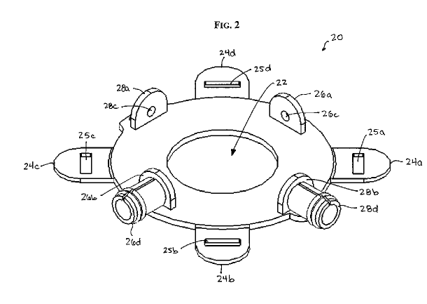

The support base 20 of the device can have a variety of configurations, shapes, and sizes, but it is preferably adapted to be positioned on a tissue surface of a patient, and to provide a stable footing for supporting the carriage assembly 40. In the illustrated embodiment, as shown in more detail in FIG. 2, the support base 20 has a generally planar, circular configuration with an opening 22 formed therethrough. The opening 22 is configured to provide access to a target tissue site, and thus it preferably has a size and shape that is sufficient to receive a tool therethrough. A person skilled in the art will appreciate that the support base 20 and the opening 22 formed therethrough can have a variety of other shapes and sizes. For example, the support base 20 and/or opening can be square, oval, rectangular, triangular, etc. The support base 20 can also be shaped to match the contour of a patient's body, or various organs. For example, the support base 20 can be substantially concave or convex to facilitate positioning at a particular location on a patient's body. The support base can, on other embodiments, be comprised of two components, a lower and standardized upper portion, wherein the lower portion comprises a range of parts, each part being shaped to match the contour of a specific part of a patient's body, and the standardized upper portion being adapted to mate to any of the various lower portion parts.

The support base 20 can also include one or more securing mechanisms or features, such as holes, slots, tabs, or adhesive stickers, etc., to secure the support base 20 to a tissue surface. As shown in FIG. 2, the support base 20 includes four tabs 24a, 24b, 24c, 24d disposed therearound. The tabs 24a-d can be used to tape the support base 20 to a tissue surface. Alternatively, or in addition, the tabs 24a-d can include slots 25a, 25b, 25c, 25d formed therein for receiving a strap or other element to secure the support base 20 onto a patient. While four tabs are shown, the support base 20 can include any number of tabs, and/or it can include a variety of other features to temporarily secure the support base 20 to a tissue surface.

As indicated above, the support base 20 is preferably configured to support the carriage assembly 40. In an exemplary embodiment, the carriage assembly 40 is movably coupled to the support base 20 by the first and second arms 30, 32. Accordingly, the support base 20 can include features for mating the first and second arms 30, 32 to the support base 20. As shown in FIG. 2, the support base 20 includes a first pair of mating elements 26a, 26b extending from and positioned on opposed sides of the support base 20 for receiving the first support arm 30, and a second pair of mating elements 28a, 28b extending from and positioned on opposed sides of the support base 20 for receiving the second support arm 32. The configuration of each mating element 26a, 26b, 28a, 28b can vary depending on the configuration of the first and second arms 30, 32, which will be discussed in more detail below, but in an exemplary embodiment the first and second pair of mating elements 26a, 26b, 28a, 28b are configured to pivotally mate the support arms 30, 32 to the support base 20. This can be achieved using, for example, a pin and bore connection. As shown in FIG. 2, each mating element 26a, 26b, 28a, 28b is in the form of an upstanding tab having a bore (only two bores 26c, 28c are shown) formed therein for receiving a corresponding pin formed on the first and second arms 30, 32. The position of each mating element 26a, 26b, 28a, 28b can also vary depending on the desired position of the first and second arms 30, 32 relative to one another. In an exemplary embodiment, as shown, the first pair of mating elements 26a, 26b are positioned 90° offset from the second pair of mating elements 28a, 28b and thus the mating elements 26a, 26b, 28a, 28b are spaced equidistantly around the perimeter of the support base 20. As a result, when the first and second arms 30, 32 are mated to the mating elements 26a, 26b, 28a, 28b, the arms 30, 32 will extend substantially transverse to one another such that they intersect one another.

As further shown in FIG. 2, two of the mating elements, e.g., mating elements 26b and 28b, can optionally include a connecting element 26d, 28d formed thereon for connecting a motor to the first and second arms 30, 32, as will be discussed in more detail below. Each connecting element 26d, 28d can have a variety of configurations, and the particular configuration can vary depending on the configurations of the motors that are used to rotate the first and second arms 30, 32 relative to the support base 20. In the illustrated embodiment, each connecting element 26d, 28d is merely a cylindrical housing or socket having an opening extending therethrough for receiving a driver on a motor, and a band formed on an external surface thereof and configured to engage and prevent rotation of the motor. The opening in each connecting element 26d, 28d is preferably aligned with the opening formed in the mating elements 26b, 28b to allow the driver on the motor to extend through the connecting elements 26d, 28d and to mate to a drive socket formed on the first and second arms 30, 32, as will be discussed in more detail below. A person skilled in the art will appreciate that various techniques can be used to connect one or more motors to the device 10, or alternatively the motor(s) can be integrally formed on or built into the device thus eliminating the need for any connecting elements. The use of external motors, however, allows the motor, which never contacts the patient, to be unclipped from the device. The device can then be discarded.

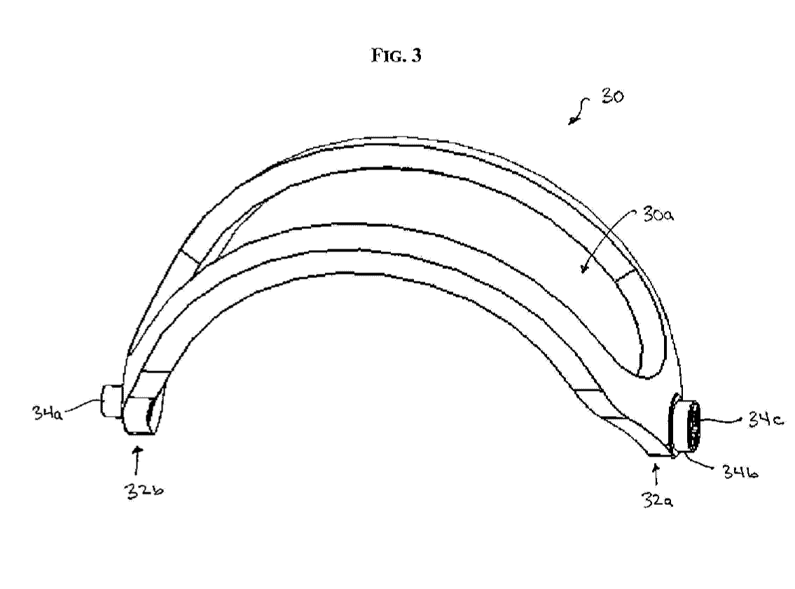

The first and second arms 30, 32 can also have a variety of configurations, but in an exemplary embodiment the arms 30, 32 are configured to pivotally couple to the support base 20, and they are adapted to hold and position the carriage assembly 40 a distance above the support base 20. FIG. 3 illustrates one of the arms 30 in more detail. As shown, the arm 30 has a generally elongate, arcuate shape with opposed ends 32a, 32b that are adapted to mate to the mating elements, e.g., elements 28a, 28b, on the support base 20. As previously discussed, in an exemplary embodiment a pin and bore connection is used to mate the arms to the connecting elements 26a, 26b, 28a, 28b on the support base 20. As shown in FIG. 3, the first and second ends 32a, 32b of the arm 30 each include a pin 34a, 34b formed thereon and adapted to extend into the bore (only one bore 28c is shown in FIG. 2) formed in the mating elements 28a, 28b on the support base 20. The second arm 32 can have a configuration that is similar to the first arm 30.

As indicated above, two of the mating elements, e.g., mating elements 26b, 28b, can include connecting elements 26d, 28d for mating to a motor. Accordingly, one of the pins on each arm 30, 32 can include a drive socket formed therein for receiving the driver on the motor connected to the connecting element. As shown in FIG. 3, pin 34b includes a drive socket 34c formed therein for receiving a driver on a motor. The drive socket 34c can have a shape, such as a hexagonal or square shape, for receiving a driver having a complementary shape. As a result, when the driver is positioned within the socket 34c, rotation of the driver will be effective to apply a rotational force to the socket 34c, thereby rotating the arm 30. Exemplary motors for rotating the arms 30, 32 will be discussed in more detail below.

Each arm 30, 32 can also include a slit or opening formed therein. The shape and size of the opening in each arm 30, 32 can vary, but in an exemplary embodiment the openings in the arms 30, 32 are configured to overlap to receive a portion of the carriage assembly 40 therethrough. Such a configuration will allow the arms 30, 32 to form a double-track for moving the carriage assembly 40 relative to the support base 20, as will be discussed in more detail below. FIG. 3 illustrates arm 30 having an elongate opening 30a extending therethrough between the first and second ends 32a, 32b thereof. When the arms 30, 32 are mated to the support 20, as shown in FIGS. 1A-1C, the openings 30a, 32a overlap and a portion of the carriage assembly 40 extends therethrough.

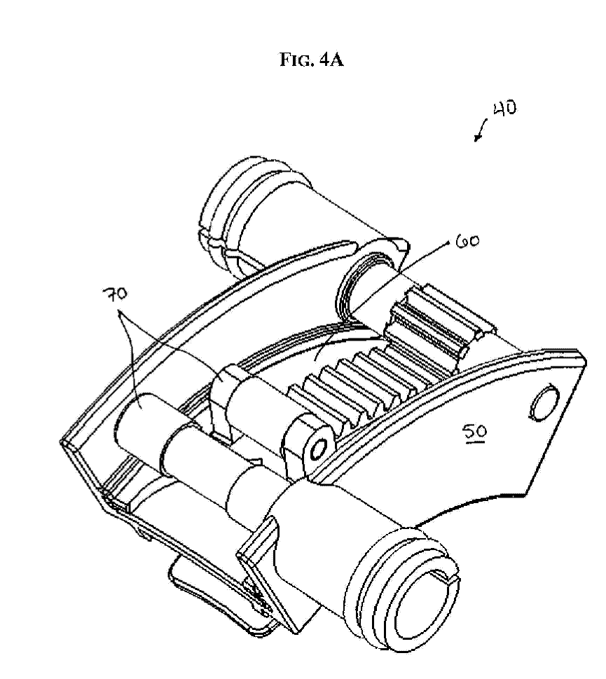

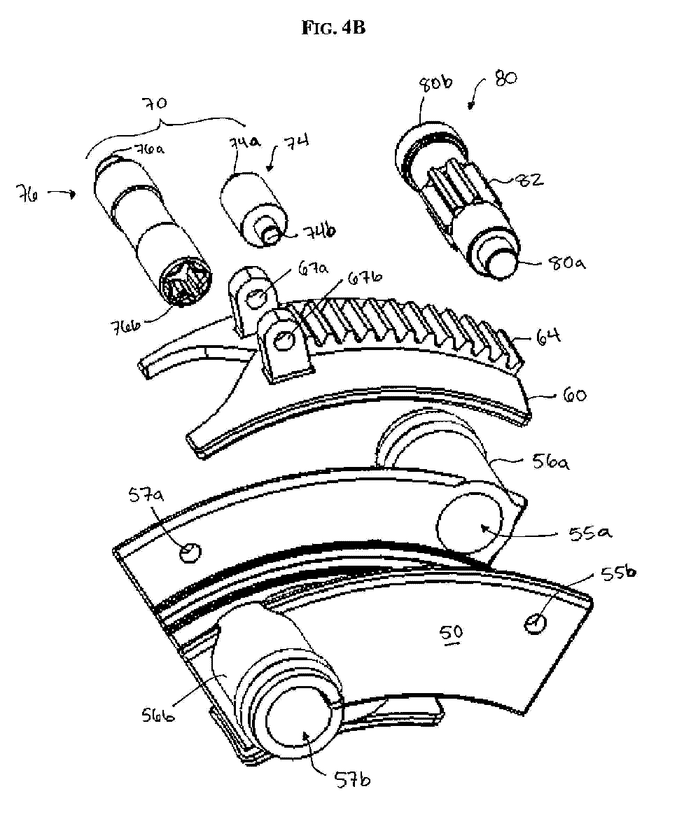

The carriage assembly 40 is shown in more detail in FIGS. 4A and 4B, and as shown the carriage assembly 40 generally includes a carriage 50 defining an opening for receiving a tool, an engagement mechanism 60 for engaging a tool extending through the opening in the carriage 50, and a driver mechanism 70 for driving a tool through the opening in the carriage 50. A person skilled in the art will appreciate that the carriage assembly 40 can have a variety of other configurations, and that various other techniques can be used to engage, position, and drive a tool into tissue.

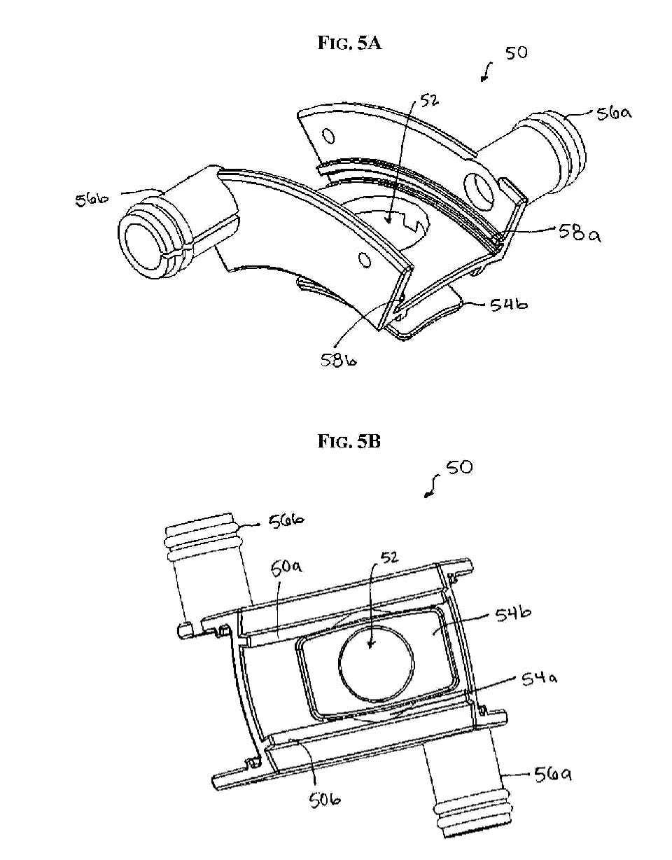

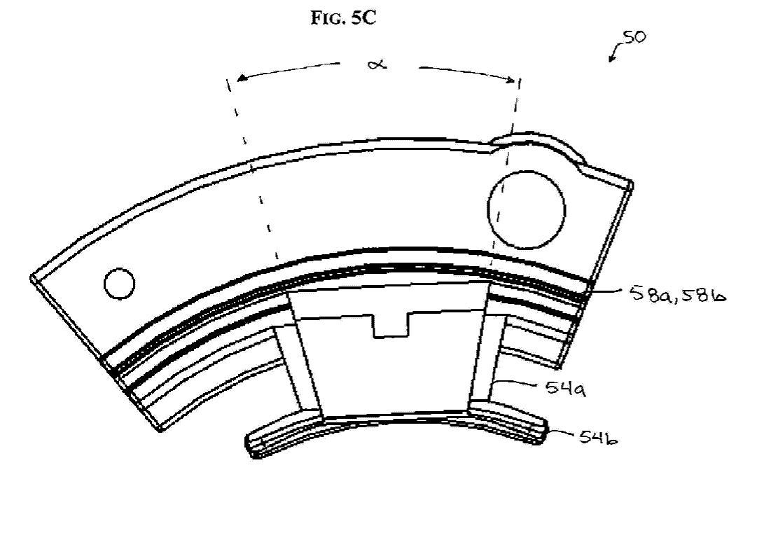

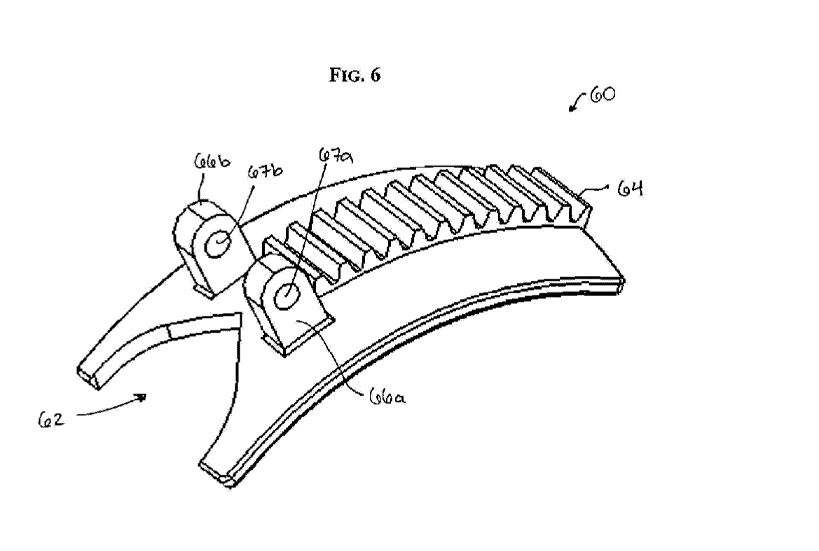

The carriage 50 is shown in more detail in FIGS. 5A-5C, and as shown the carriage 50 is in the form of a housing having a generally arcuate bottom surface with opposed sidewalls extending therefrom. The arcuate bottom surface of the carriage 50 allows the carriage 50 to slide along one of the support arms, e.g., the first support arm 30 as shown in FIGS. 1A-1C, and the sidewalls allow the carriage 50 to slidably receive the engagement mechanism 60 for engaging a tool extending through the carriage 50. As further shown in FIGS. 5A and 5B, the carriage 50 includes an opening or viewing window 52 extending through the bottom surface thereof for receiving a tool. The viewing window 52 can extend through a hollow housing 54a that is coupled to the bottom surface of the carriage 50, and that forms an extension on the carriage 50. The hollow housing 54a allows the carriage 50 to be coupled to the first and second support arms 30, 32. In particular, the housing 54a can extend through the openings 30a, 32a in the first and second support arms 30, 32 to allow the carriage 50 to be engaged and moved in response to movement of the support arms 30, 32. The particular shape of the hollow housing 54a can vary depending on the shape of the openings 30a, 32a in the arms 30, 32, as well as the size and shape of a tool to be inserted therethrough. In an exemplary embodiment, the housing 54a has a shape and a size that is sufficient to allow a variety of tools of various sizes to be inserted therethrough. The shape and size of the housing 54a is also preferably configured to allow a tool to pivot within the viewing window 52, thus loosely retaining a tool in a generally upright position while allowing the tool to be moved and manipulated. The viewing window 52 can also have a size that allows the device 10 to be removed over a tool implanted within a patient. In the embodiment shown in FIG. 5C, the housing 54a is generally cone-shaped and defines a conical range of motion within which a tool can pivotally move. The angle α of the cone can vary depending on the desired range of motion and the particular tool being used, but in one exemplary embodiment the cone angle α is in the range of 10 to 15°.

As further shown in FIGS. 5A-5C, the carriage 50 can also include a flange 54b formed around a terminal end of the housing 54a. The flange 54b can be configured to retain or lock the carriage 50 on the support arms 30, 32, preventing accidental disengagement during use of the device. The flange 54b can also have a shape that allows the carriage 50 to be easily disassembled and removed from the supports arms 30, 32, if desired. As shown in FIGS. 5A and 5C, the flange 54b is substantially rectangular. Such a configuration allows the flange 54b to be inserted at an angle through the openings 30a, 32a in the supports arms 30, 32 when the openings 30a, 32a are aligned with one another, and then one of the arms, e.g., the second arm 32, can be rotated 90° to lock the carriage 50 onto the arms 30, 32. The arms 30, 32 can then be slightly compressed and snapped between the first and second pair of mating elements 26a, 26b, 28a, 28b on the support base 20.

The carriage 50 can also include rails 50a, 50b formed on an inferior or bottom surface thereof for facilitating positioning of the carriage 50 relative to the support arms 30, 32. In particular, the carriage 50 is preferably configured to rest on one of the arms, e.g. the first arm 30. The rails 50a, 50b can extend along the bottom surface of the carriage 50, and they can be configured to be positioned within the opening 30a in the first arm 30, or around the first arm 30. The rails 50a, 50b can prevent rotation of the carriage 50 relative to the first arm 30, thereby preventing the carriage 50 from being removed. The rails 50a, 50b can also facilitate sliding movement of the carriage 50 along the first arm 30.

As further shown in FIGS. 5A and 5C, the carriage 50 can further include tracks 58a, 58b formed therein for slidably receiving the engagement mechanism 60, which is adapted to engage a tool extending through the viewing window 52 in the carriage 50. The illustrated tracks 58a, 58b are in the form of opposed rails that extend along internal sidewalls of the carriage 50, and that define a groove for slidably seating the engagement mechanism 60.

The carriage 50 can also include other features, such as first and second connecting elements 56a, 56b for mating a motor to a driver 80 for moving the engagement mechanism 60 and a driver 70 for advancing a tool, as will be discussed below. The first and second connecting elements 56a, 56b can be similar to the connecting elements 26d, 28d on the support base 20, and in particular they can be in the form of substantially cylindrical housings having a bore extending therethrough for receiving a driver on a motor.

An exemplary engagement mechanism 60 is shown in more detail in FIG. 6, and as shown it has a generally elongate shape with an arcuate profile. The arcuate shape allows the engagement mechanism 60 to slidably seat within the tracks 58a, 58b formed in the carriage 50, and to move between an open position (shown in FIG. 1B), in which the engagement mechanism 60 is spaced a distance apart from the viewing window 52 to provide access to the viewing window 52, and a closed position (shown in FIG. 1C), in which the engagement mechanism 60 extends over the viewing window 52 to engage a tool extending through the viewing window. To facilitate engagement of a tool extending through the viewing window 52, the engagement mechanism 60 can include a cut-out 62 formed therein and adapted to receive a tool. While the cut-out 62 can have a variety of shapes of sizes, in one exemplary embodiment, as shown in FIG. 6, the cut-out is substantially triangular. Such a shape will allow the cut-out 62 to urge a tool, such as a needle, into horizontal alignment with the cut-out 62.

As explained above, the engagement mechanism 60 is preferably movable between an open and closed position. Slidable movement of the engagement mechanism 60 can be achieved using a variety of techniques, but in one exemplary embodiment the carriage 50 includes a driver 80 formed thereon and adapted to slide the engagement mechanism 60 within the tracks 58a, 58b formed in the carriage 50. One exemplary driver 80 is shown in FIGS. 4A and 4B, and as shown the driver 80 is in the form of a shaft having teeth or gears 82 formed thereon. The gears 82 are configured to mate to corresponding teeth or gear, i.e., a rack 64, formed on a surface of the engagement mechanism 60. As a result, rotation of the driver 80 in a first direction is effective to slide the engagement mechanism 60 in a first direction within the carriage 50, and rotation of the driver 80 in an opposite direction is effective to slide the engagement mechanism 60 in a second, opposite direction within the carriage 50, thereby moving the engagement mechanism 60 between the open and closed positions. The driver 80 can be rotated using a motor, as will be described in more detail below. Preferably, opposed ends 80a, 80b of the driver 80 are configured to be rotatably positioned within opposed openings 55a, 55b formed in the carriage 50. One of the ends, e.g., end 80b, can include a drive socket formed therein for receiving a driver on a motor. The drive socket can be similar to drive socket 34c previously described with respect to FIG. 3. In use, when the driver 80 is coupled to the carriage 50, a motor can be coupled to the connecting element 56a to allow a driver on a motor to be positioned through the connecting element 56a and to be coupled to the drive socket on the driver 80. Actuation of the motor will rotate the driver, thereby rotating the drive socket and thus the driver 80 to move the engagement mechanism 60.

As previously explained, the carriage assembly 40 can also include a driver 70 for driving a tool through the viewing window 52 and into tissue. FIGS. 4A and 4B illustrate one exemplary embodiment of a driver 70 that includes a passive roller 74 mated to the engagement mechanism 60, and an active roller 76 mated to the carriage 50. The active roller 76 is configured to be rotated by a driver to drive a tool through the viewing window 52, and the passive roller 74 is configured to rotate as a result of movement of the tool relative thereto, as will be discussed below. The rollers 74, 76 can be mounted on the engagement mechanism 60 and carriage 50 using a variety of techniques, but in an exemplary embodiment each roller 74, 76 includes opposed ends that are configured to be received within corresponding openings or bores. In particular, the active roller 76 can include opposed ends 76a, 76b that extend into corresponding bores 57a, 57b which are formed in opposed sidewalls of the carriage 50. The passive roller 74 can include opposed ends 74a, 74b that extend into corresponding bores 67a, 67b formed in the engagement mechanism 60. The bores 67a, 67b, which are shown in FIGS. 4B and 6, are formed in opposed arms 66a, 66b formed on and extending from a surface of the engagement mechanism 60. The opposed arms 66a, 66b are positioned adjacent to the cut-out 62 to allow the passive roller 74 to be positioned adjacent to the drive roller 76 when the engagement mechanism 60 is in the closed position. As a result, a tool extending through the viewing window 52 will be engaged between the passive and active rollers 74, 76.

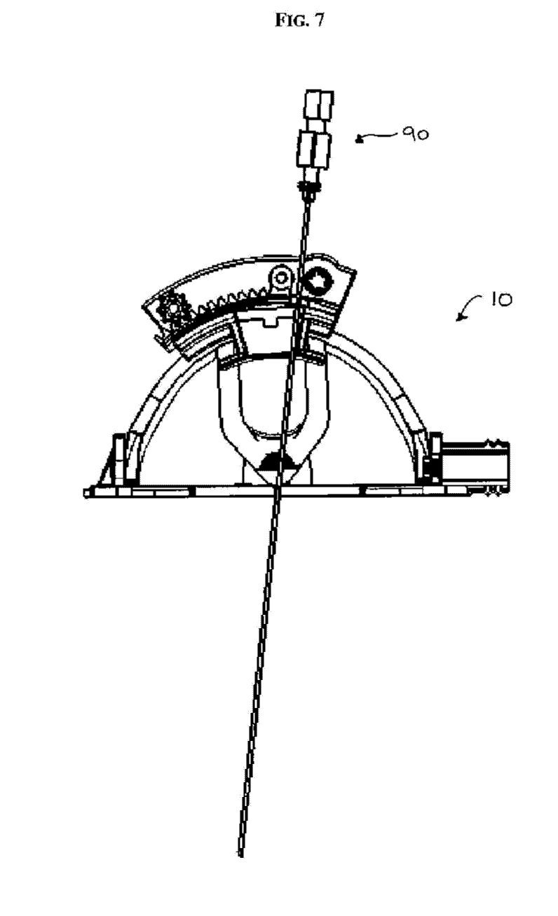

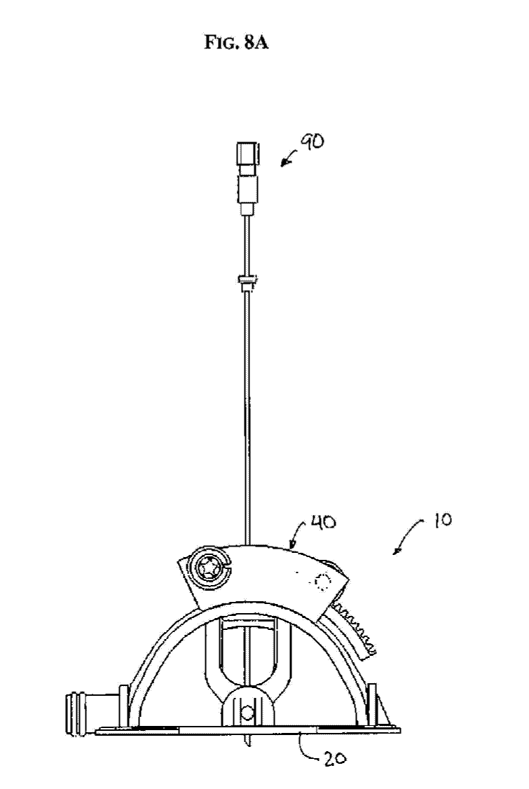

In order to actuate the driver 70, one end of the active roller 76, e.g., end 76b, can include a socket formed therein and configured to receive a driver on a motor. The socket can be similar to socket 34c previously described with respect to FIG. 3. In use, when the active roller 76 is coupled to the carriage 50, a motor can be mated to the connecting element 56b such that the driver on the motor extends through the connecting element and into the socket formed in the drive roller 76. As a result, when the motor is actuated the driver will rotate to rotate the socket, thereby rotating the drive roller 76. The drive roller 76 will thus cause a tool positioned between the drive roller 76 and the passive roller 74 to advance into tissue. The drive roller 76 can also be driven in a reverse direction to retract the tool from tissue. FIG. 7 illustrates the device 10 having a tool, in the form of a biopsy needle 90, extending therethrough. As shown, the engagement mechanism 60 is in the closed position such that the tool 90 is positioned within the cut-out portion in the engagement mechanism 60, and is engaged between the passive and active rollers 74, 76.

In order to facilitate movement of a tool, the drive roller and/or the passive roller 74, 76 can optionally include a protective member, such as rubber, disposed around at least a portion thereof. The protective member can be effective to decrease the contact stresses between the roller(s) 74, 76 and the tool, and it can improve traction between the roller(s) 74, 76 and the tool as the tool is driven into or out of tissue.

As indicated above, a motor can be used to rotate the arms 30, 32 relative to the support base 20 and thereby position the viewing window 52 in the carriage assembly 40 at a desired orientation. A motor can also be used to slide the engagement mechanism 60 within the carriage 50, thereby engaging and disengaging a tool extending through the viewing window 52. A motor can further be used to rotate the active roller 76 to advance and retract a tool extending through the viewing window and engaged between the active roller 76 and the passive roller 74 in the engagement mechanism 60. In an exemplary embodiment a separate motor is configured to removably mate to each connecting element 26d, 28d, 56a, 56b to allow each motor to be individually actuated to control movement of the arms 30, 32, movement of the engagement mechanism 60, and actuation of the driver 70 for advancing and retracting a tool. Such a removable connection is particularly advantageous as it allows the entire device 10 to be formed from a disposable material, such as a polymer. The motors, which do not contact the patient's skin, can simply be detached from the device 10 after use and the device 10 can be discarded.

While various types of motors can be used to perform each of task, one exemplary type of motor that can be used is a step motor, which is an electromagnetic, rotary actuator that mechanically converts digital pulse inputs to incremental shaft rotation. With the appropriate logic, step motors can be bidirectional, synchronous, provide rapid acceleration, stopping, and reversal, and will interface easily with other digital mechanisms. As a result, the motors can allow for accurate and precise control of each movement. One exemplary step motor is a series AM 1020 Motor with a planetary gear head and a 256:1 reduction. The step angle of the motor is 18° thus allowing an angular position resolution of 0.07° to be obtained. The motor also has the ability to orientate a tool positioned within the viewing window 52 by moving the arms 30, 32 at a speed of about 360°/s, and to drive a tool into tissue at a rate of about 20 mm/s. In other embodiments, a DC motor, hydraulics, battery power, or other techniques can be used to actuate the drivers and rotate the arms.

In another embodiment, the device 10 can include markers in the support base 20, support arms 30, 32, and/or carriage assembly 40. The markers can be formed from a radiopaque material to allow the markers to be viewed in an image of the patient to create reference points in a coordinate system, that, for example, may include the patient, target surgical site, device, and/or imaging apparatus. The reference points can be used to facilitate automatic targeting of the tool to the target surgical site. For example, the device 10 may contain metallic parts in the support base 20 that would appear in a CT image, allowing one skilled in the art to calculate the correct input parameters for the device 10 to insert the tool to the target surgical site.

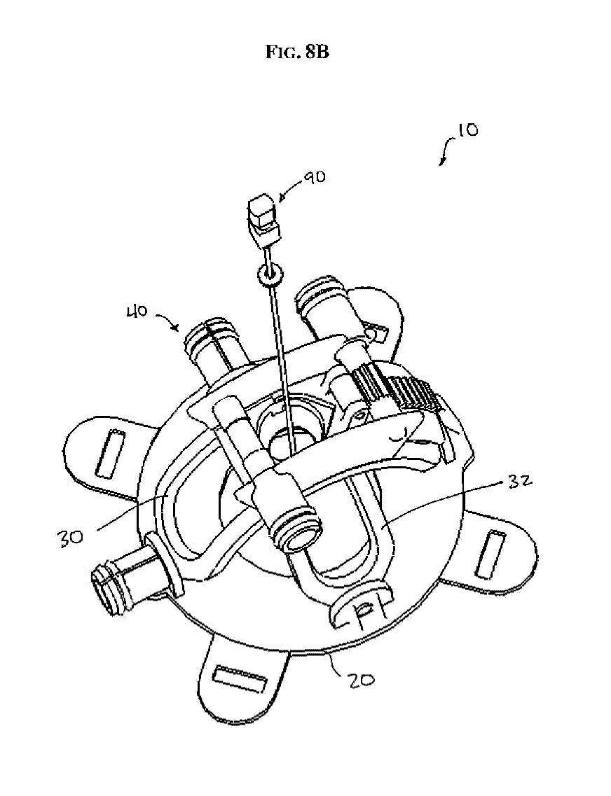

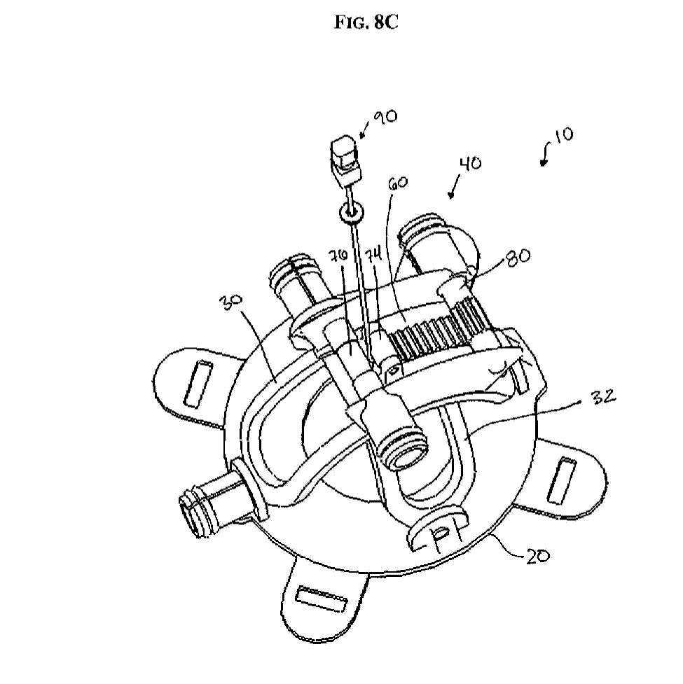

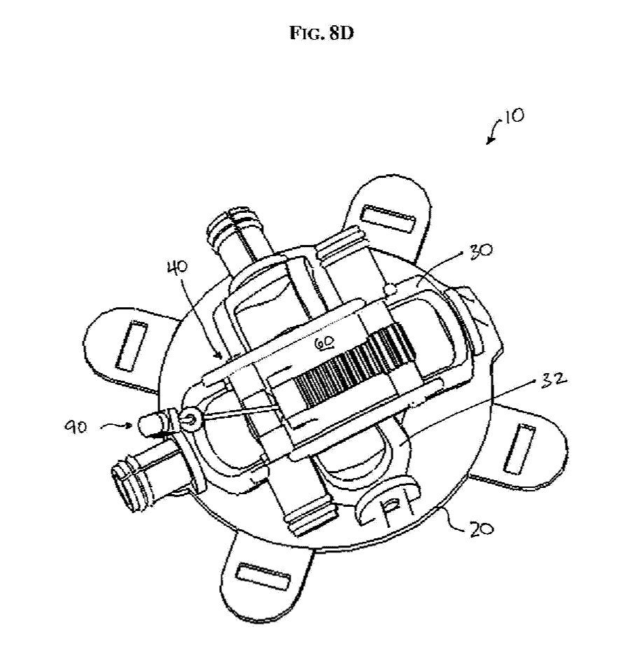

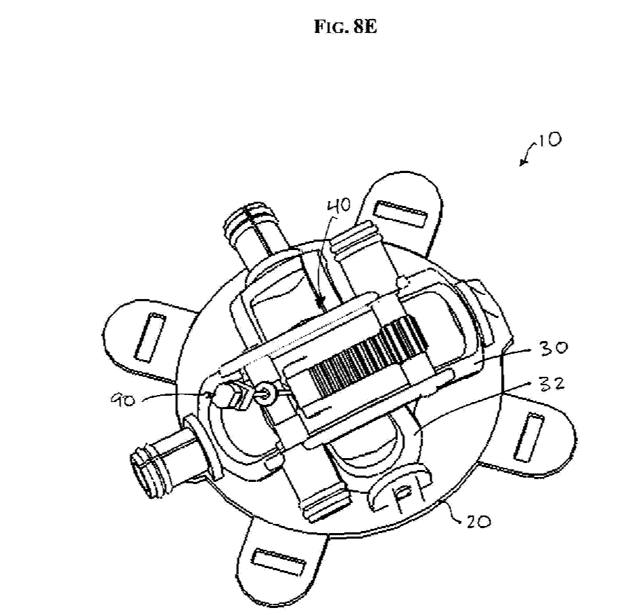

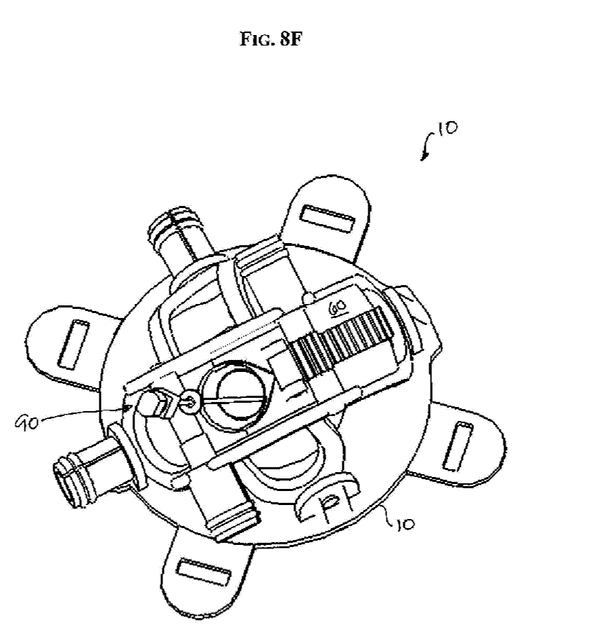

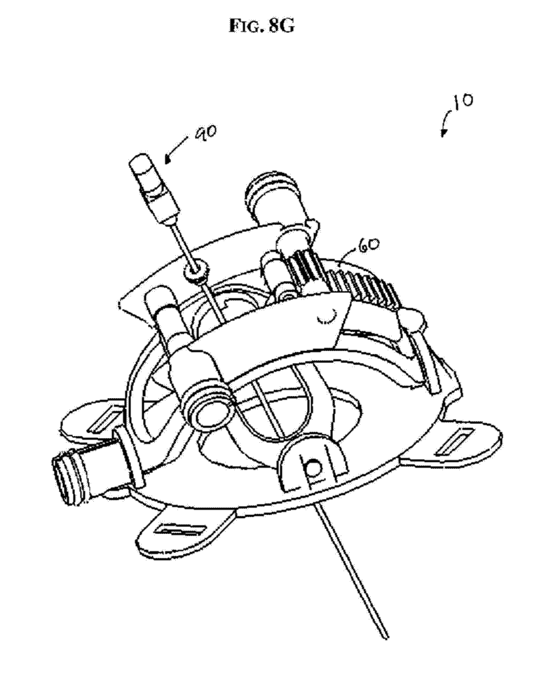

FIGS. 8A-8G illustrate an exemplary method for using the device 10. While the device 10 can be used to guide and insert a variety of tools into tissue, in one exemplary embodiment the device 10 is used to guide and insert a biopsy needle into tissue to remove a sample for analysis. The device 10 can be positioned directly on a tissue surface (not shown) of a patient at a location above a target surgical site, such as a tumor. Since the device 10 is positioned on the patient, the device will move with the patient, for example with the rise and fall of the patient's chest during breathing. A motor can be connected to each connecting element 26d, 28d, 56a, 56b on the device 10, as previously discussed. The motors can be coupled to a control box, which in turn can be connected to a computer to allow the motors to be remotely actuated. The device 10 can be secured to the patient using the tabs 24a, 24b, 24c, 24d on the support base 20 and straps, adhesives, etc. Once the device 10 is secured, a biopsy needle 90 can be positioned through the viewing window 52 in the carriage assembly 40, as shown in FIG. 8A. The engagement mechanism 60 will be in the open position to provide access to the viewing window 52. A distal tip of the needle 90 is then positioned at the insertion point, and it can be at least partially penetrated into the tissue. In this position, the needle 90 is free to pivot within the viewing window 52, as shown in FIG. 8B.

In an exemplary embodiment, the device 10 can be used with an imaging method and system, such as computed tomography (CT), magnetic resonance imaging (MRI), ultrasound, X-ray, X-ray fluoroscopy, etc. The device 10 can be formed from a non-metallic material, such as plastic, so that the device 10 does not interfere with the image, and it can be sized to fit within the confines of, for example, a CT or MRI machine. A plastic device with MRI-compatible motors, such as a piezoelectric motor, can be used safely in an MRI machine's strongly magnetic environment. Once positioned on the patient, the operator can exit the room and operate the device remotely from a control room containing the computer for controlling the motors. The patient can be scanned with the device 10 and needle 90 positioned on the patient, and an image can be viewed. While simultaneously viewing the image, the user can control the needle's insertion angle and depth remotely through an intuitive interface. As shown in FIG. 8C, the motor coupled to the engagement mechanism 60 can be actuated to rotate the driver 80 and thereby slide the engagement mechanism 60 to the closed position. This will cause the cut-out portion on the engagement mechanism 60 to engage and orient the needle 90 with the cut-out portion, and to position the needle 90 between the passive and active rollers 74, 76. Either prior to or after closing the engagement mechanism 60, the motor attached to each of the first and second arms 30, 32 can be individually actuated to rotate the first and second arms 30, 32 to a desired angular orientation relative to the support base 20. As the first and second arms 30, 32 pivot relative to the support base 20, the carriage assembly 40 will move, as shown in FIG. 8D, thereby positioning the needle 90 at a desired angular orientation. The particular angular orientation of the needle 90 can be confirmed using the imaging apparatus, and if necessary, the arms 30, 32 can be further moved to reposition the carriage assembly 40 and thus the needle 90 until a desired angular orientation is achieved.

Once the needle 90 is properly oriented at a desired insertion angle, the motor coupled to the active roller 76 can be actuated to rotate the active roller 76, thereby driving the needle 90 into the tissue, as shown in FIG. 8E. The motor can be used to control the insertion speed and/or depth, which can be viewed using the imaging apparatus. The device can optionally include feedback for assessing unusual torque loads on the motor and to stop needle insertion when the torque load exceeds a predetermined load. The insertion depth can also be controlled using a stop formed on the needle 90 to limit the insertion depth of the needle. For example, a proximal portion of the needle 90 can include a flange disposed therearound and configured to abut against the passive and active rollers 74, 76. A clamp could also be used to function as a depth stop. In other embodiments, a camera or other imaging mechanism can be mounted directly on the device or needle to facilitate viewing of the needle relative to the target surgical site. The needle 90 could also optionally include depth markings formed thereon to indicate the insertion depth.

After the needle 90 is fully inserted to obtain a tissue sample from a target surgical site, the driver can optionally be actuated to rotate the active roller 76 in an opposite direction, thereby removing the needle 90 from the tissue. Alternatively, the needle 90 can remain deployed and the engagement mechanism 60 can be moved to the open position to allow a surgeon to remove the needle 90, as shown in FIGS. 8F and 8G. This is particularly advantageous as the needle 90 can be loosely retained by the device 10, and still remain penetrated within the patient. The needle 90 can thus move with the patient or any internal organs, thus reducing the risk of damaging internal tissues or organs. The device 10 can also optionally be removed before the needle 90 is removed from the patient by sliding the device 10 over the needle 90. A person skilled in the art will appreciate that the steps can be performed in virtually any order and may vary depending on the procedure being performed.

As previously indicated, a software interface can optionally be used in conjunction with the imaging apparatus and the device 10 to control positioning of the needle 90. For example, the software can be configured to receive the insertion angle and to command the device to attain that position. Alternatively, the software can be configured to determine the insertion angle and to command the device to attain that angle. Small “jogs” are also possible, allowing the tool to be moved in increments, such as 5°. The user inputs are converted into desired rotations and speed and sent to the controller which in turn sends step commands to the individual motor drivers. The needle insertion depth can similarly be controlled. In other embodiments, the device 10 can include a joystick or wand for actuating the device.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A guidance and insertion system, comprising:
a support base adapted to be placed on a tissue surface;
a carriage movably mounted on the support base and defining a viewing window therethrough for receiving a tool; and
a driver coupled to the carriage and adapted to drive a tool through the viewing window and into tissue positioned beneath the support base.

2. The system of claim 1, wherein the carriage is movably mounted to the support base by first and second support arms that are movably coupled to the support base.

3. The system of claim 2, further comprising a first drive socket formed on the first support arm and adapted to couple to a motor for moving the first support arm relative to the support base, and a second drive socket formed on the second support arm and adapted to couple to a motor for moving the second support arm relative to the support base.

4. The system of claim 2, wherein the first and second support arms are rotatably coupled to the support base.

5. The system of claim 2, wherein the first support arm extends substantially transverse to the second support arm.

6. The system of claim 2, wherein the first and second support arms each include an opening formed therein, and wherein a portion of the carriage extends through the openings in the first and second support arms such that movement of the first and second support arms relative to one another is effective to move the carriage relative to the support base.

7. The system of claim 6, wherein each support arm has a substantially arcuate shape, and wherein the first and second support arms extend substantially transverse to one another such that the openings overlap to receive a portion of the carriage.

8. The system of claim 1, wherein the carriage includes an engagement mechanism slidably disposed thereon and adapted to slidably move to engage a tool extending through the viewing window.

9. The system of claim 8, wherein the engagement mechanism includes a cut-out formed therein and adapted to seat a tool extending through the viewing window, the cut-out being configured to urge the tool into a predetermined position.

10. The system of claim 8, further comprising a driver rotatably coupled to the carriage and adapted to rotate to slide the engagement mechanism within a track formed in the carriage.

11. The system of claim 1, wherein the driver comprises at least one roller adapted to rotate to drive a tool through the viewing window.

12. The system of claim 11, wherein the at least one roller comprises an active roller adapted to couple to a motor for rotating the roller, and a passive roller, the active and passive rollers being configured to engage a tool positioned therebetween and extending through the viewing window.

13. The system of claim 12, wherein the active roller is coupled to the carriage, and the passive roller is coupled to an engagement mechanism that is slidably disposed on the carriage and that is adapted slidably move to engage a tool extending through the viewing window.

14. The system of claim 1, wherein the support base includes a plurality of securing mechanisms formed thereon and adapted to facilitate securing of the support base to a patient.

15. The system of claim 1, wherein the system is at least partially formed from a polymeric material.

16. A method for guiding and inserting a tool into tissue, comprising:
positioning a guide system on a tissue surface of a patient such that the guide system is positioned over a target surgical site;
positioning a tool through a viewing window of the guide system;
actuating the guide system to engage the tool positioned within the viewing window;
actuating the guide system to adjust a trajectory of the tool based on an image of the guide system and the target surgical site; and
actuating a driver mechanism on the guide system to advance the tool into tissue.

17. The method of claim 16, wherein positioning the tool further comprises penetrating a distal portion of the tool into tissue.

18. The method of claim 16, wherein actuating the guide system to adjust a trajectory of the tool comprises pivoting at least one arm pivotally coupled to a support base of the guide system, the at least one arm having a carriage mounted thereon and defining the viewing window extending therethrough.

19. The method of claim 18, wherein engaging the tool comprises slidably moving an engagement mechanism disposed on the carriage to a closed position.

20. The method of claim 19, wherein the tool is positioned between an active roller and a passive roller in the engaged position, and wherein actuating a driver mechanism to advance the tool comprises rotating the active roller.

21. The method of claim 16, wherein an image of the guide system and the target surgical site is viewed using computer tomography.

22. The method of claim 16, wherein the guide system and driver mechanism are actuated remotely.

23. The method of claim 16, wherein the guide system is secured to the tissue surface using a mating element.

24. The method of claim 16, wherein the tissue is a target surgical site.
